(19) United States
(12) Patent Application Publication
(10) Pub. No.: US 2026/0174999 A1
(43) Pub. Date: Jun. 25, 2026

(54) CATHETER SYSTEM INCLUDING A GUIDEWIRE HAVING A COIL SPRING PORTION

(71) Applicant: Becton, Dickinson and Company, Franklin Lakes, NJ (US)

(72) Inventors: Curtis H. Blanchard, Herriman, UT (US); Weston F. Harding, Lehi, UT (US); Benjamin Parker Hopwood, Salt Lake City, UT (US)

(21) Appl. No.: 18/990,931

(22) Filed: Dec. 20, 2024

(51) Int. Cl. A61M 25/09

(52) U.S. Cl. CPC A61M 25/09; A61M 2025/09058

(57) ABSTRACT

A catheter system may include a catheter adapter and a catheter extending distally from a distal end of the catheter adapter. The catheter system may include a needle hub and an introducer needle having a proximal end fixed within the needle hub. The catheter system may include a guidewire having a proximal end, a distal end, and a coiled spring portion. The guidewire may be disposed within the introducer needle, and the coiled spring portion may be proximal to the distal end of the catheter. The catheter system may include a guidewire hub proximal to the needle hub. The coiled spring portion may be configured to compress in response to the distal end of the guidewire contacting a blood vessel and decompress in response to entry of the guidewire into the blood vessel.

### BACKGROUND

Catheters are a ubiquitous part of hospital care and are used as a main access point to draw blood, infuse medications, and assess bodily function. Catheters can include peripheral intravenous catheters, midline catheters, arterial catheters, or other classes of catheters.

As its name implies, a catheter that is “over-the-needle” may be mounted over an introducer needle having a sharp distal tip. The catheter and the introducer needle can be inserted at a shallow angle through the skin and into vasculature of the patient. In order to verify proper placement of the introducer needle and the catheter in the vasculature, a clinician may confirm there is flashback of blood in a flashback chamber of the catheter system or in a space between the introducer needle and the catheter. After placement of the introducer needle within the vasculature has been confirmed, the clinician may remove the introducer needle, leaving the catheter in place in the vasculature for future blood draw or fluid infusion.

Difficult venous access results in an inability of the clinician to place the catheter within the vasculature on a first attempt. Up to half of peripheral intravenous catheter insertions may experience this challenge of difficult venous access. Difficult venous access is exacerbated by veins that are fragile, small, deep, or tortuous and may result from heavy drug use, high Body Mass Index (BMI), or other conditions.

Guidewires can assist in catheter placement within the vasculature, particularly when there is difficult venous access. One known method of using a guidewire includes the Seldinger technique in which a needle is inserted into a blood vessel. A guidewire is then inserted through the needle. The needle is then removed, and a catheter is advanced over the guidewire.

A guidewire can also be integrated into a catheter system and used to guide a catheter of the catheter system. An example of a catheter system having an integrated guidewire is the POWERGLIDE™ Midline Catheter available from Becton, Dickinson and Company of Franklin Lakes, New Jersey.

Both the Seldinger technique and catheter systems like the POWERGLIDE™ Midline Catheter with integrated guidewires provide the benefit of threading the catheter over a guidewire already in place in the vasculature. Advantageously, the guidewire can grip a wall of a vein or artery and give support to enable the catheter to penetrate the wall. Also, when the guidewire is already in place in the vasculature, and the catheter accesses the vasculature, the catheter is less likely to cause damage to the vasculature or cause infiltration or extravasation. However, the Seldinger technique and catheter systems like the POWERGLIDE™ Midline Catheter with integrated guidewires often require significant action by the clinician in order to place the guidewire within the vasculature, and guidewires used are typically much longer than the catheter being placed.

The subject matter claimed herein is not limited to embodiments that solve any disadvantages or that operate only in environments such as those described above. Rather, this background is only provided to illustrate one example technology area where some implementations described herein may be practiced.

### SUMMARY

In some embodiments, a catheter system may include a catheter adapter, which may include a distal end and a proximal end. In some embodiments, the catheter system may include a catheter, which may include a distal end. In some embodiments, the catheter may extend distally from the distal end of the catheter adapter.

In some embodiments, the catheter system may include an introducer needle, which may include a proximal end and a sharp distal tip. In some embodiments, the introducer needle may extend through the catheter. In some embodiments, the catheter system may include a needle hub. In some embodiments, the proximal end of the introducer needle may be fixed within the needle hub.

In some embodiments, the catheter system may include a guidewire, which may include a proximal end, a distal end, and a coiled spring portion between the proximal end of the guidewire and the distal end of the guidewire. In some embodiments, the guidewire may be disposed within the introducer needle.

In some embodiments, a guidewire hub may be proximal to the needle hub. In some embodiments, the proximal end of the guidewire may be fixed within the guidewire hub. In some embodiments, the coiled spring portion may be configured to compress in response to the distal end of the guidewire contacting a blood vessel and decompress in response to entry of the guidewire into the blood vessel.

In some embodiments, the coiled spring portion may be proximal to the sharp distal tip. In some embodiments, the coiled spring portion may be proximal to the proximal end of the introducer needle. In some embodiments, the coiled spring portion may be disposed within the guidewire hub.

In some embodiments, the distal end of the guidewire may be distal to sharp distal tip. In some embodiments, the distal end of the guidewire may be configured to retract proximal to the sharp distal tip in response to compression of the coiled spring portion.

In some embodiments, the guidewire hub may be coupled to the needle hub. In some embodiments, in response to uncoupling of the guidewire hub and the needle hub and proximal movement of the guidewire hub with respect to the needle hub, the distal end of the guidewire may be configured to retract proximal to the sharp distal tip.

In some embodiments, a proximal end of the needle hub may include a first cutout, and a distal end of the guidewire hub may include a second cutout. In some embodiments, the first cutout and the second cutout may be proximate each other to form a pocket having a U-shape configured to receive a thumb or finger of a user.

In some embodiments, the guidewire hub may be coupled to the needle hub in a slip fit. In some embodiments, a distal end of the guidewire hub may include a cylindrical recess. In some embodiments, a proximal end of the needle hub may include a cylindrical protrusion that is disposed within the cylindrical recess.

In some embodiments, the catheter system may include a needle safety housing coupled to the proximal end of the catheter adapter. In some embodiments, the catheter system may include a needle safety clip disposed within the needle safety housing and configured to shield the sharp distal tip when the sharp distal tip is withdrawn proximally past the needle safety clip.

In some embodiments, the guidewire may include a marking. In some embodiments, the catheter system may include a transparent window. In some embodiments, in response to the coiled spring portion being compressed, the marking is configured to move into or out of the transparent window.

In some embodiments, the distal end of the guidewire may be straight. In some embodiments, the distal end of the guidewire may include a coil distal to the sharp distal tip.

In some embodiments, the guidewire may include a feature. In some embodiments, the catheter system may include a stop contacting the feature to prevent distal movement of the guidewire. In some embodiments, in response to compression of the coiled spring portion, the stop may be removed from contact with the feature, and the coiled spring portion may be configured to decompress.

In some embodiments, the distal end of the guidewire may be distal to the sharp distal tip a first distance. In some embodiments, in response to the stop being removed from contact with the feature and the coiled spring portion decompressing, the distal end of the guidewire may be distal to the sharp distal tip a second distance. In some embodiments, the second distance may be greater than the first distance.

In some embodiments, the guidewire hub may include a slot. In some embodiments, the proximal end of the guidewire may be within the guidewire hub. In some embodiments, the catheter system may include a guidewire slider extending through the slot and coupled to the guidewire. In some embodiments, the guidewire slider may be movable along the slot between a proximal position and a distal position.

In some embodiments, the guidewire slider may be coupled to the guidewire proximal to the coiled spring portion. In these and other embodiments, when the guidewire slider is in the proximal position, the distal end of the guidewire may be proximal to the sharp distal tip. In some embodiments, the proximal end of the guidewire may be secured to the guidewire slider.

In some embodiments, the guidewire slider may be coupled to the guidewire distal to the coiled spring portion. In these and other embodiments, when the guidewire slider is in the proximal position, the coiled spring portion may be compressed and the distal end of the guidewire may be proximal to the sharp distal tip. In some embodiments, the proximal end of the guidewire may be secured within a wall of the guidewire hub.

It is to be understood that both the foregoing general description and the following detailed description are examples and explanatory and are not restrictive of the invention, as claimed. It should be understood that the various embodiments are not limited to the arrangements and instrumentality shown in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE SEVERAL VIEWS OF THE DRAWINGS

Example embodiments will be described and explained with additional specificity and detail through the use of the accompanying drawings in which:

FIG. 1A is an upper perspective view of an example catheter system, according to some embodiments;

FIG. 1B is a partially exploded view of the catheter system of FIG. 1B, according to some embodiments;

FIG. 1C is a perspective view of an example guidewire hub having an example guidewire fixed within the guidewire hub, according to some embodiments;

FIG. 1D is an upper perspective view of an example coiled spring portion, according to some embodiments;

FIG. 1E is another partially exploded view of the catheter system of FIG. 1B, according to some embodiments;

FIG. 1F is a cross-sectional view of the catheter system of FIG. 1B, according to some embodiments;

FIG. 1G is a cross-sectional view of the catheter system of FIG. 1B, illustrating the coiled spring portion in an example compressed position, according to some embodiments;

FIG. 1H is an upper perspective view of an example distal end of the catheter system of FIG. 1B when the coiled spring portion in the compressed position, according to some embodiments;

FIG. 2A is an upper perspective view of an example transparent window of the catheter system, illustrating an example marking within the transparent window, according to some embodiments;

FIG. 2B is an upper perspective view of the transparent window of the catheter system, illustrating the coiled spring portion in the compressed position and the marking of FIG. 2A, according to some embodiments;

FIG. 2C is an upper perspective view of the transparent window of the catheter system, illustrating an example marking within the transparent window, according to some embodiments;

FIG. 2D is an upper perspective view of the transparent window of the catheter system, illustrating the coiled spring portion in the compressed position and the marking of FIG. 2C, according to some embodiments;

FIG. 3 is an upper perspective view of the distal end of an example catheter system, illustrating an example coil of an example guidewire, according to some embodiments;

FIG. 4A is a cross-sectional view of an example catheter system, illustrating an example stop contacting an example feature to prevent distal movement of the guidewire, also illustrating an example coil spring portion compressed a first amount, according to some embodiments;

FIG. 4B is a cross-sectional view of the catheter system of FIG. 4A, illustrating the coil spring portion compressed a second amount, according to some embodiments;

FIG. 4C is a cross-sectional view of the catheter system of FIG. 4A, illustrating the coil spring portion decompressed, according to some embodiments;

FIG. 5A is a cross-sectional view of an example catheter system having an example guidewire slider, illustrating the guidewire slider in an example proximal position, according to some embodiments;

FIG. 5B is a cross-sectional view of an example catheter system having an example guidewire slider, illustrating the guidewire slider in an example proximal position, according to some embodiments; and

FIG. 5C is a cross-sectional view of the catheter system of FIG. 5B, illustrating the guidewire slider in an example distal position, according to some embodiments;

FIG. 6A is an upper perspective view of an example catheter system having an example guidewire slider, illustrating the guidewire slider in an example proximal position;

FIG. 6B is a cross-sectional view of the catheter system of FIG. 6A, illustrating the guidewire slider in the proximal position, according to some embodiments;

FIG. 7A is an upper perspective view of an example catheter system having an example guidewire slider, illustrating the guidewire slider in an example proximal position; and

FIG. 7B is a cross-sectional view of the catheter system of FIG. 7A, illustrating the guidewire slider in an example proximal position, according to some embodiments.

### DESCRIPTION OF EMBODIMENTS

For clarity it is to be understood that the word “proximal” refers to a direction relatively closer to a clinician using the catheter system described in the present disclosure, and the word “distal” refers to a direction relatively further from the clinician using the catheter system described in the present disclosure. For example, the end of a catheter placed within the body of a patient is considered a distal end of the catheter, while the catheter end remaining outside the body is considered a proximal end of the catheter. Also, the words “including,” “has,” and “having,” as used in the present disclosure, including the claims, shall have the same meaning as the word “comprising.”

In some embodiments, a catheter system 10 may include a catheter adapter 12, which may include a distal end 14 and a proximal end 16. In some embodiments, the catheter system 10 may include a catheter 18 having a distal end 19 and a proximal end. In some embodiments, the proximal end of the catheter 18 may be secured within the catheter adapter 12. In some embodiments, the catheter 18 may extend distally from the distal end 14 of the catheter adapter 12. In some embodiments, the catheter 18 may be a peripheral intravenous catheter (PIVC), a midline catheter, a peripherally-inserted central catheter, an arterial catheter, or another type of catheter.

In some embodiments, the catheter system 10 may include an introducer needle 20, which may include a proximal end 22 and a sharp distal tip 24. In some embodiments, the sharp distal tip 24 may facilitate insertion of the catheter 18 threaded over the introducer needle 20 through skin and tissue of a patient and into vasculature of the patient. In some embodiments, the introducer needle 20 may extend through the catheter 18 such that the sharp distal tip 24 is positioned distal to the catheter 18 to facilitate insertion of the catheter system 10 into the patient. In some embodiments, the sharp distal tip 24 may include a bevel surrounding a distal opening of the introducer needle 20.

In some embodiments, the catheter system 10 may include a needle hub 26. In some embodiments, the proximal end 22 of the introducer needle 20 may be fixed within the needle hub 26. In some embodiments, the catheter system 10 may include a guidewire 28, which may be disposed within or extend through the introducer needle 20. In some embodiments, the guidewire 28 may include a proximal end 30, a distal end 32, and a coiled spring portion 34 between the proximal end 30 of the guidewire 28 and the distal end 32 of the guidewire 28.

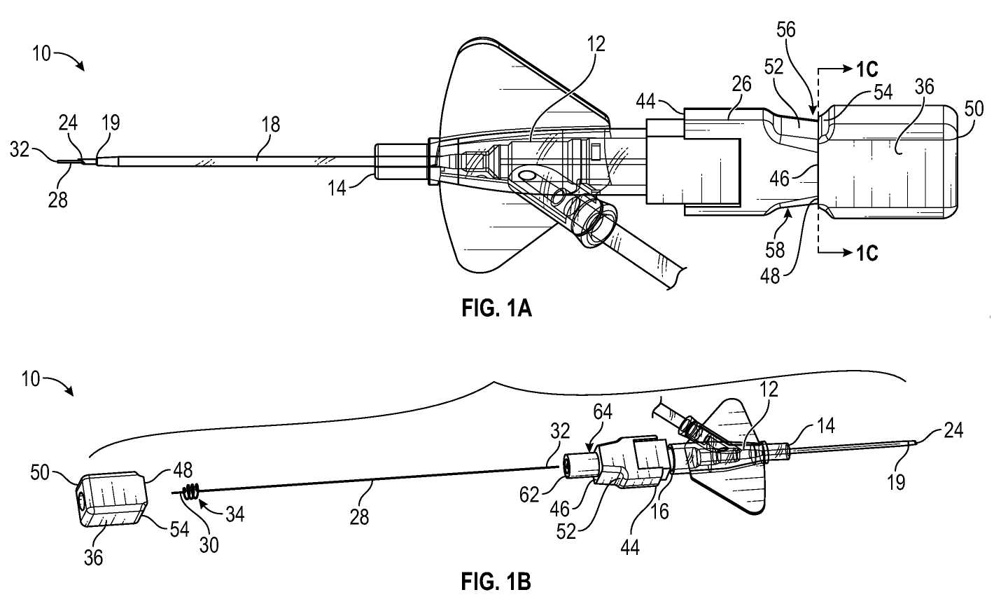

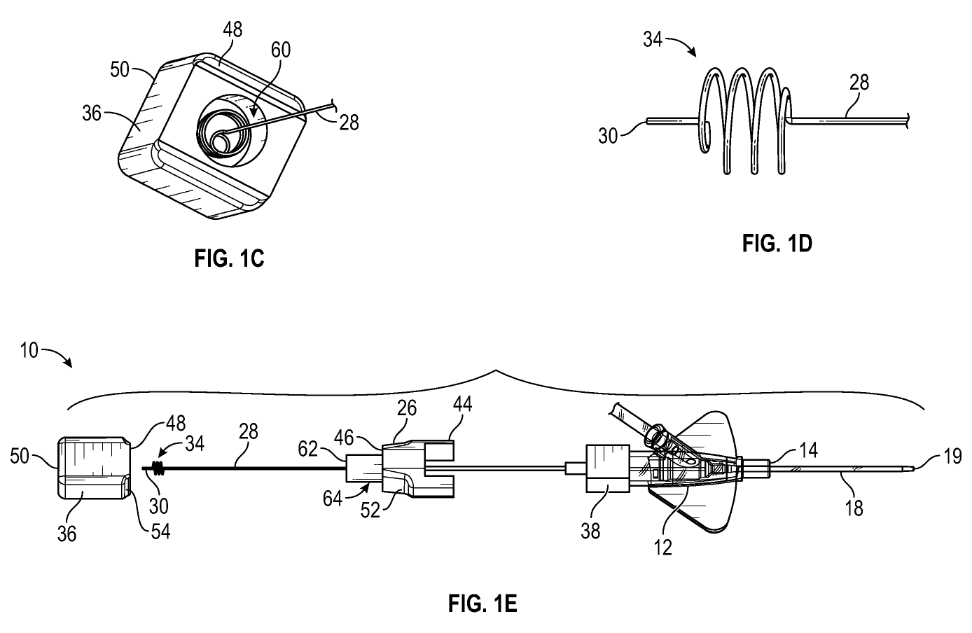

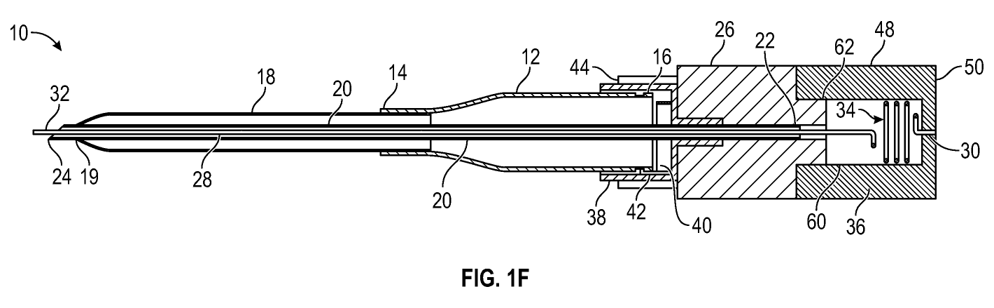

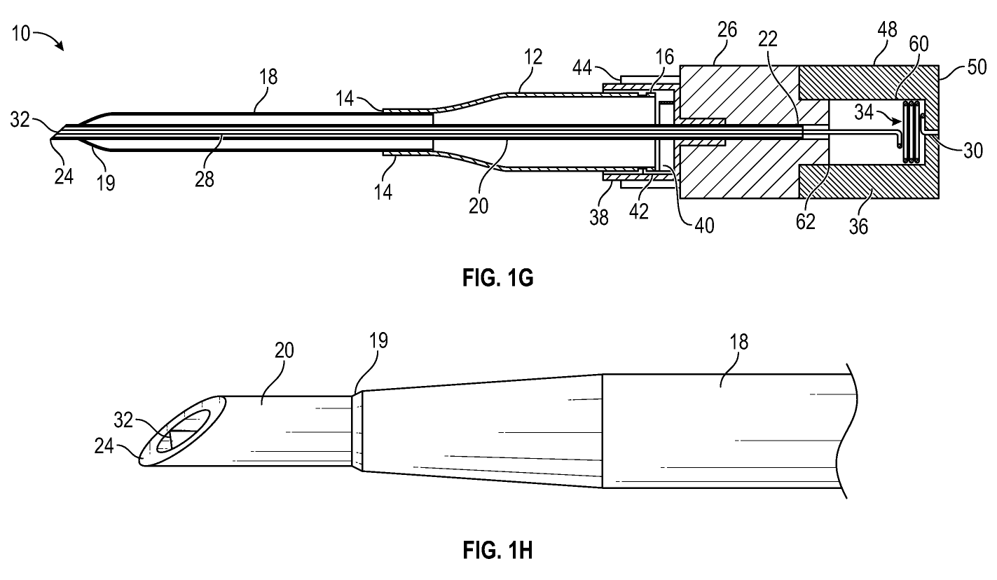

In some embodiments, a guidewire hub 36 may be proximal to the needle hub 26. In some embodiments, the guidewire hub 36 may include a housing that includes the proximal end 30 within the housing. In some embodiments, the proximal end 30 of the guidewire 28 may be fixed within or stationary with respect to the guidewire hub 36. In some embodiments, the distal end 32 of the guidewire 28 may be distal to the distal end 19 of the catheter 18, as illustrated, for example, in FIG. 1A and 1F. In some embodiments, the catheter system 10 in FIGS. 1A and 1F may correspond to a resting configuration or an assembled configuration of the catheter system 10. In some embodiments, the catheter system 10 in FIGS. 1A and 1F may correspond to an insertion configuration ready for insertion into the patient.

In some embodiments, the distal end 32 of the guidewire 28 extending a short distance beyond the sharp distal tip 24 of the introducer needle 20 as in FIGS. 1A and 1F may facilitate the guidewire 28 retracting proximally into the introducer needle 20 during insertion into the patient due to compression of the coiled spring portion 34 when the distal end 32 contacts the skin, the tissue, or a wall of the vasculature of the patient. As an example, the short distance could be between 1-4 mm for a peripheral vein or another suitable distance. As another example, the short distance could be greater than 1-4 mm for an artery or another suitable distance. In some embodiments, the distal end 32 of the guidewire 28 may retract fully into the introducer needle 20 during insertion into the patient due to compression of the coiled spring portion 34 when the distal end 32 contacts the skin, the tissue, and a wall of the vasculature of the patient.

In some embodiments, the coiled spring portion 34 may be compressed, as illustrated, for example, in FIGS. 1G-1H, until the introducer needle 20 penetrates the vasculature, such as a vein or artery, opening a path with little or no resistance to the guidewire 28 such that the coiled spring portion 34 quickly expands or decompresses. In some embodiments, when the introducer needle 20 penetrates the vasculature, the sharp distal tip 24 has moved through the wall of the vasculature and the sharp distal tip 24 is in the lumen of the vasculature, allowing the coiled spring portion 34 to decompress. Thus, in some embodiments, the coiled spring portion 34 may facilitate insertion of the introducer needle 20 and the catheter 18 into the patient without interference from the distal end 32 of the guidewire that is positioned inside the introducer needle 20 during movement of the introducer needle 20 through the skin, the tissue, and the wall of the vasculature.

Advantageously, in some embodiments, when the coiled spring portion 34 decompresses in response to the introducer needle 20 having penetrated the vasculature, the guidewire 28 extending beyond the sharp distal tip 24 may act as a guide for the catheter 18, facilitating insertion of the catheter 18 into the vasculature. In some embodiments, the guidewire 28 extending beyond the distal end 19 of the catheter 18 when the introducer needle 20 enters the lumen of the vasculature may allow the guidewire 28 to grip the wall of the vasculature to stabilize the catheter system 10 within the vasculature and avoid sliding of the introducer needle 20.

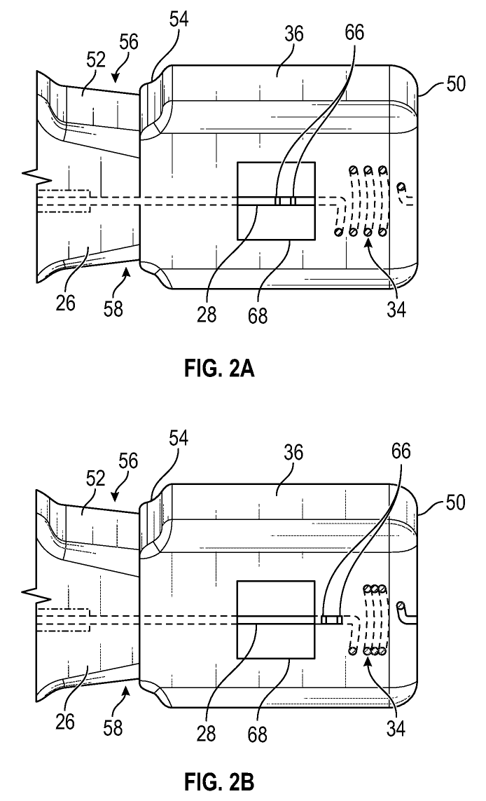

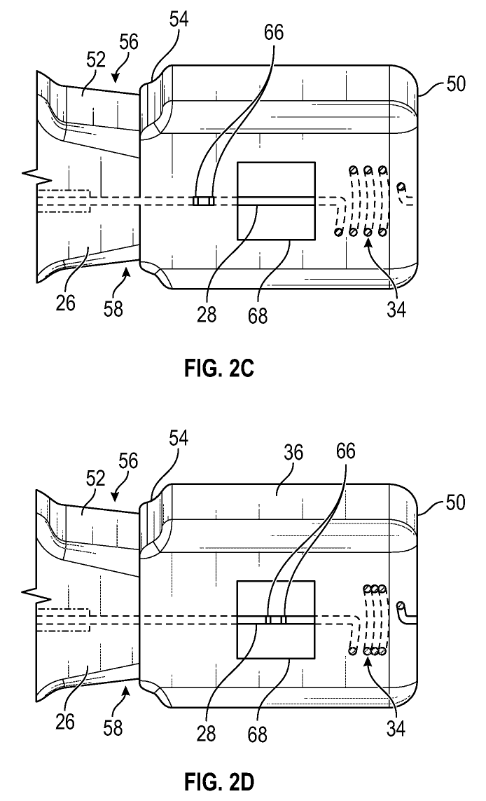

In some embodiments, in response to the introducer needle 20 penetrating the vasculature, the coiled spring portion 34 may decompress distally or back to its initial state in the resting or assembled configuration. In some embodiments, decompression or spring forward of the coiled spring portion 34 when the introducer needle 20 penetrates the vasculature may provide a tactile signal to the clinician that the introducer needle 20 has entered the vasculature. In some embodiments, the tactile signal may include a pop of the coiled spring portion 34 when it springs forward. In some embodiments, the catheter system 10 may be configured to provide a visual signal (such as, for example, a color or light) and/or an audible signal (such as, for example, a buzzer) in response to decompression of the coiled spring portion 34 when the introducer needle 20 penetrates the vasculature. In some embodiments, the visual signal may include one or more markings 66 and/or a transparent window 68, as further described with respect to FIGS. 2A-2D.

In some embodiments, if an obstacle is encountered after the sharp distal tip 24 is positioned in the lumen of the vasculature, the coiled spring portion 34 may again compress, similar to compression when the distal end 32 of the guidewire moves through the skin, the tissue, and the wall of the vasculature. In some embodiments, compression of the coiled spring portion 34 when the distal end 32 of the guidewire 28 contacts the obstacle may provide some protection to the wall of the vasculature and enable additional attempts for the clinician to advance the introducer needle 20 and the catheter 18 within the vasculature.

In some embodiments, compression of the coiled spring portion 34 when the distal end 32 of the guidewire 28 contacts the obstacle may provide another tactile signal to the clinician. In some embodiments, compression of the coiled spring portion 34 when the distal end 32 contacts the obstacle may provide another visual signal (such as, for example, a color or light) and/or another audible signal (such as, for example, a buzzer). In some embodiments, in response to one or more of the other tactile signal, the other visual signal, and the other audible signal, the clinician may make adjustments in angle or advancement speed in order to facilitate proper insertion of the catheter 18 within the vasculature.

In some embodiments, the coiled spring portion 34 may facilitate passive use of the guidewire 28 by the clinician, because the distal end 32 of the guidewire 28 may be positioned distal to the catheter 18 in the assembled configuration and may automatically retract into the introducer needle 20 when resistance is encountered. Thus, the guidewire 28 may require less action by the clinician compared to the Seldinger technique and/or catheter systems like the POWERGLIDE™ Midline Catheter with integrated guidewires. Moreover, in some embodiments, because the distal end 32 of the guidewire 28 is positioned beyond the catheter 18 and the introducer needle 20 in the assembled configuration, the guidewire 28 can be shorter than guidewires used with the Seldinger technique and/or guidewires integrated into catheter systems like the POWERGLIDE™ Midline Catheter. In some embodiments, a short guidewire may allow an entirety of the catheter system 10 to be short, which may improve the ergonomics of the catheter system 10 to facilitate ease of insertion of the catheter 18 into the patient.

In some embodiments, due to distal end 32 of the guidewire 28 extending the short distance beyond the sharp distal tip 24 as opposed to a long distance, the guidewire 28 may be constructed of stainless steel rather than nitinol. In some embodiments, the guidewire 28 may be constructed nitinol or another suitable material.

In some embodiments, the catheter system 10 may include a needle safety housing 38. As illustrated, for example, in FIGS. 1A-1B and 1F-1G, the needle safety housing 38 may be coupled to the proximal end 16 of the catheter adapter 12. In some embodiments, the catheter system 10 may include a needle safety clip 40 disposed within the needle safety housing 38 and configured to shield the sharp distal tip 24 when the sharp distal tip 24 is withdrawn proximally past the needle safety clip 40. In some embodiments, the needle safety clip 40 may include a V-clip, which may include a pawl 42 that may engage a groove in the catheter adapter 12 to prevent the needle safety housing 38 from separating from the catheter adapter 12 until the introducer needle 20 is withdrawn far enough proximally to activate the V-clip, as further described in U.S. Patent Application Publication No. 2024/0157096, filed November 6, 2023, entitled “CATHETER SYSTEM HAVING A GUIDEWIRE,” which is hereby incorporated by reference in its entirety.

In some embodiments, the guidewire hub 36 may be coupled to the needle hub 26 as illustrated, for example, FIGS. 1A-1B and 1F-1G. In some embodiments, in response to uncoupling of the guidewire hub 36 and the needle hub 26 and proximal movement of the guidewire hub 36 with respect to the needle hub 26, the distal end 32 of the guidewire 28 may be configured to retract proximal to the sharp distal tip 24 of the introducer needle 20. Thus, in some embodiments, the clinician may manually retract the guidewire 28 by uncoupling the guidewire hub 36 and the needle hub 26 and moving the guidewire hub 36 proximally with respect to the needle hub 26. This manual retraction of the guidewire 28 may be useful if decompression or spring forward of the coiled spring portion 34 occurs outside of the vasculature before reaching the lumen of the vasculature due to, for example, levering of the sharp distal tip 24 of the introducer needle 20 outside of the vasculature. In some embodiments, after manually retracting the guidewire 28 by uncoupling the guidewire hub 36 and the needle hub 26 and moving the guidewire hub 36 proximally with respect to the needle hub 26, the clinician may then move the guidewire hub 36 distally and couple the guidewire hub 36 and the needle hub 26 together such that decompression or spring forward of the coiled spring portion 34 can happen again. In some embodiments, when the guidewire 28 retracts proximal to the sharp distal tip 24, the distal end 32 may be entirely inside the introducer needle 20.

In some embodiments, when there is resistance encountered during insertion of the introducer needle 29 and the catheter 18 into the patient (such as when the sharp distal tip 24 of the introducer needle 29 is in the skin, the tissue, or the wall of the vasculature), and as a result the coiled spring portion 34 compresses, the clinician may need to apply a force (such as by gripping) to hold the guidewire hub 36 and the needle hub 26 together to keep the guidewire hub 36 and the needle hub 26 coupled and prevent them from separating. In some embodiments, when the resistance is encountered during insertion of the introducer needle 29 and the catheter 18 into the patient, and as a result the coiled spring portion 34 compresses, if the clinician does not apply the force, the guidewire hub 36 may automatically move proximally away from the needle hub 26, and the distal end 32 of the guidewire 28 may move proximally into the introducer needle 20. Thus, in some embodiments, the catheter system 10 may facilitate passive withdrawal of the guidewire 28 in response to removal of the force applied by the clinician, which may be useful if the vasculature is not penetrated successfully on a first attempt so the guidewire 28 is not distal to the distal end 19 of the catheter 18 prior to starting a second attempt.

In some embodiments, the needle hub 26 may include a distal end 44 and a proximal end 46, and the guidewire hub 36 may include a distal end 48 and a proximal end 50. In some embodiments, the proximal end 46 of the needle hub 26 may include a first cutout 52, and the distal end 48 of the guidewire hub 36 may include a second cutout 54. In some embodiments, the first cutout 52 and the second cutout 54 may be proximate each other to form a pocket 56 having a U-shape configured to receive a thumb or finger of a user, which may facilitate tactile feedback to the clinician, including minor distal or proximal position changes of the guidewire hub 36. In some embodiments, the U-shape may be generally U-shaped having ends higher than a middle portion.

In some embodiments, when there is resistance encountered during insertion of the introducer needle 29 and the catheter 18 into the patient (such as when the sharp distal tip 24 of the introducer needle 29 is in the skin, tissue, or wall of the vasculature), and as a result the coiled spring portion 34 compresses, the finger pressure within the U-shape may help the clinician keep the guidewire hub 36 coupled to the needle hub 26. In some embodiments, when the resistance is encountered during insertion of the introducer needle 29 and the catheter 18 into the patient, and as a result the coiled spring portion 34 compresses, if the clinician releases the finger pressure from the U-shape, the guidewire hub 36 may automatically move proximally away from the needle hub 26, and the distal end 32 of the guidewire 28 may move proximally into the introducer needle 20. Thus, in some embodiments, a size and shape of the needle hub 26 and the guidewire hub 36 forming the pocket 56 may facilitate passive withdrawal of the guidewire 28 in response to removal of the finger pressure, which may be useful if the vasculature is not penetrated successfully on the first attempt so the guidewire 28 is not distal to the distal end 19 of the catheter 18 prior to starting the second attempt.

In some embodiments, the needle hub 26 and the guidewire hub 36 may form another pocket 58 similar to the pocket 56 and opposite the pocket 56. In some embodiments, the pocket 56 and the other pocket 58 may facilitate a pinching action by the clinician to hold the needle hub 26 and the guidewire hub 36 together such that the needle hub 26 and the guidewire hub 36 are coupled.

In some embodiments, the guidewire hub 36 may be coupled to the needle hub 26 in a slip fit, which may facilitate easy separation between the guidewire hub 36 and the needle hub 26 in order to manually retract the guidewire 28 by moving the guidewire hub 36 proximally with respect to the needle hub 26 or allow passive withdrawal of the guidewire 28 in response to removal of the finger pressure. In these embodiments, the distal end 48 of the guidewire hub 36 may include a cylindrical recess 60. In some embodiments, a proximal end 46 of the needle hub 26 may include a cylindrical protrusion 62 that is disposed within the cylindrical recess 60 when the guidewire hub 36 and the needle hub 26 are coupled together. In some embodiments, the audible signal may include a click or tap noise created in response to the coiled spring portion 34 decompressing or springing forward and contacting a portion of the catheter system 10, such as, for example, the cylindrical protrusion 62.

In some embodiments, compression of the coiled spring portion 34 in response to the distal end 32 of the guidewire 28 contacting the skin, the tissue, or the wall of the vasculature may be in a proximal direction due to the guidewire hub 36 and the needle hub 26 being coupled together. In some embodiments, decompression in response to the introducer needle 20 penetrating the vasculature may be in distal direction due to the guidewire hub 36 and the needle hub 26 being coupled together. In some embodiments, when the guidewire hub 36 and the needle hub 26 are coupled together, the cylindrical protrusion 62 may be inserted into the cylindrical recess 60 a maximum distance and/or a stepped surface 64 of the needle hub 26 may contact the distal end 48 of the guidewire hub 36.

In some embodiments, when the guidewire hub 36 and the needle hub 26 are coupled together, they may be engaged in a snap fit or another suitable fit. In some embodiments, the clinician may be able to manually withdraw the guidewire 28 or passively withdraw the guidewire 28 by, for example, removing the finger pressure, prior to the guidewire hub 36 and the needle hub 26 being engaged in the snap fit. In some embodiments, the guidewire hub 36 and the needle hub 26 may be snapped together to remove the introducer needle 20 and the guidewire 28 from the catheter system 10 at a same time when the catheter 18 is in place within the vasculature.

As illustrated, for example, in FIG. 1C, in some embodiments, the proximal end 30 of the guidewire 28 may be bonded within the cylindrical recess 60 or another portion of the guidewire hub 36. In some embodiments, the coiled spring portion 34 may be proximal to the distal end 19 of the catheter 18 and/or disposed within the guidewire hub 36 such that the coiled spring portion 34 does not contact the skin, the tissue, or the wall of the vasculature during insertion. In some embodiments, the coiled spring portion 34 may be disposed entirely within the guidewire hub 36 and/or may be proximal to the proximal end 22 of the introducer needle 20, which may allow the coiled spring portion 34 to have an outer diameter greater than an inner diameter of the introducer needle 20, shortening a length of the coiled spring portion 34. In some embodiments, the coiled spring portion 34 may be disposed in both the cylindrical recess 60 and the cylindrical protrusion 62 inserted into the cylindrical recess 60. As illustrated, for example, in FIG. 1D, in some embodiments, the coiled spring portion 34 may include several loops of coil to facilitate retraction into the introducer needle 20 in response to compression of the coiled spring portion 34.

In some embodiments, an outer surface of the guidewire 28 may be spaced apart from an inner surface of the introducer needle 20 forming a lumen of the introducer needle to facilitate flashback of blood between the outer surface of the guidewire 28 and the inner surface of the introducer needle 20. In some embodiments, decompression or spring forward of the coiled spring portion 34 when the introducer needle 20 penetrates the vasculature may provide a faster signal, such as a tactile, visual, and/or audible signal, than the flashback of blood, which may reduce a risk of transfixing the vein or the artery.

Referring now to FIGS. 2A-2B, in some embodiments, the guidewire 28 may include one or more markings 66. In some embodiments, the catheter system 10 may include a transparent window 68. In some embodiments, the transparent window 68 may include glass, which may be magnifying, or another transparent material. In some embodiments, the transparent window 68 including glass or another transparent material may prevent any fluid within the catheter system 10 from leaking out of the catheter system 10. In some embodiments, the transparent window 68 may include an opening with no material therein, which may facilitate visibility of the markings 66.

In some embodiments, in response to the catheter system 10 being in the resting configuration or the assembled configuration, the markings 66 may be visible to the clinician within the transparent window 68, as illustrated, for example, in FIG. 2A. In some embodiments, in response to the coiled spring portion 34 being compressed in the proximal direction, as illustrated, for example, in FIG. 2B, the markings 66 may be configured to move out of the transparent window 68, indicating to the clinician the sharp distal tip 24 is disposed within the skin, the tissue, the wall of the vasculature, or encountering the obstacle. In some embodiments, movement of the markings 66 back into the transparent window 68 when the coiled spring portion 34 decompresses may signal to the clinician that the sharp distal tip 24 has penetrated the vasculature and is disposed within the lumen of the vasculature.

In some embodiments, the transparent window 68 may be disposed within the guidewire hub 36 or another suitable portion of the catheter system 10. In some embodiments, the transparent window 68 may be a separate piece from a body of the guidewire hub 36 and may be inserted and secured within the body. In other embodiments, the transparent window 68 may be monolithically formed as a single unit with the body of the guidewire hub 36, which may be entirely transparent or may include a transparent portion forming the transparent window 68.

Referring now to FIGS. 2C-2D, in some embodiments, in response to the catheter system 10 being in the resting configuration or the assembled configuration, the markings 66 may be outside the transparent window such that they are not visible to the clinician, as illustrated, for example, in FIG. 2C. In some embodiments, in response to the coiled spring portion 34 being compressed in the proximal direction, as illustrated, for example, in FIG. 2D, the markings 66 may be configured to move into the transparent window 68, indicating to the clinician the sharp distal tip 24 is disposed within the skin, the tissue, the wall of the vasculature, or encountering an obstacle. In some embodiments, movement of the markings 66 back out of the transparent window 68 when the coiled spring portion 34 decompresses may signal to the clinician that the sharp distal tip 24 has penetrated the vasculature and is disposed within the lumen of the vasculature.

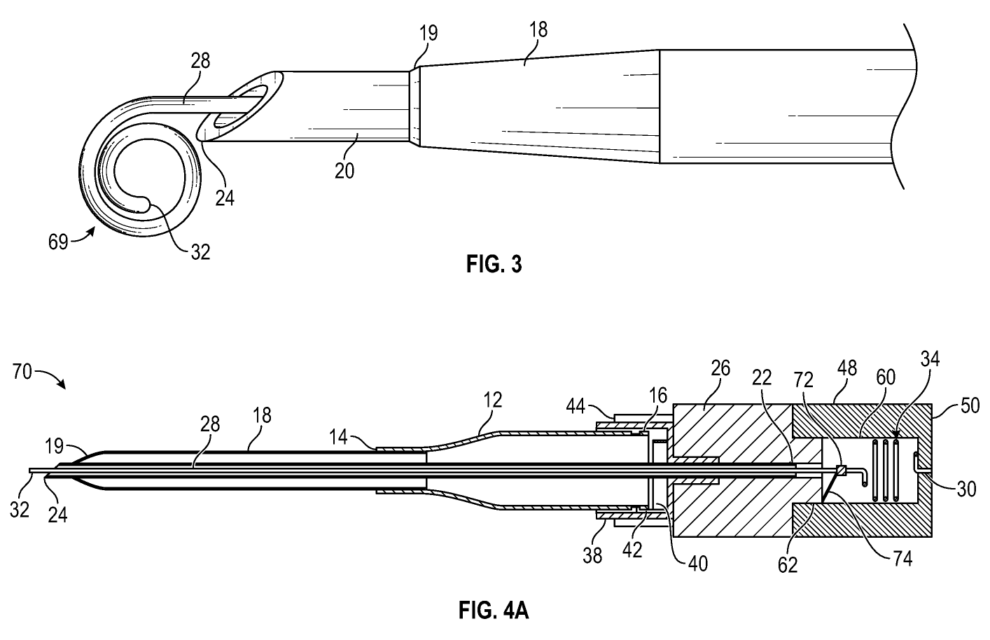

Referring now to FIG. 3, in some embodiments, the distal end 32 of the guidewire 28 may include a coil 69 distal to the sharp distal tip 24. In other embodiments, the distal end 32 of the guidewire 28 may be straight. In some embodiments, a portion of the guidewire 28 proximate the coiled spring portion 34 may be straight to facilitate insertion through the introducer needle 20.

As illustrated, for example, in FIG. 3, in some embodiments, the coil 69 may loop from approximately a central axis of the introducer needle 20 downwardly past the sharp distal tip 24 to cover the sharp distal tip 24 when the catheter system 10 is in the resting configuration or the assembly configuration. In some embodiments, the coil 69 covering the sharp distal tip 24 during insertion of the introducer needle 20 and the catheter 18 into the patient may reduce a risk of the sharp distal tip 24 spearing through or transfixing the vasculature. In some embodiments, the coil 69 may include a curly Q that is flat and curls around an axis generally perpendicular to the central axis of the introducer needle 20. In some embodiments, the curly Q may facilitate covering an area immediately distal to the sharp distal tip 24 with multiple layers of the guidewire 28. In some embodiments, the sharp distal tip 24 may include a sharp edge or point forming a distalmost surface of the introducer needle 20 and configured to first contact the skin of the patient during insertion.

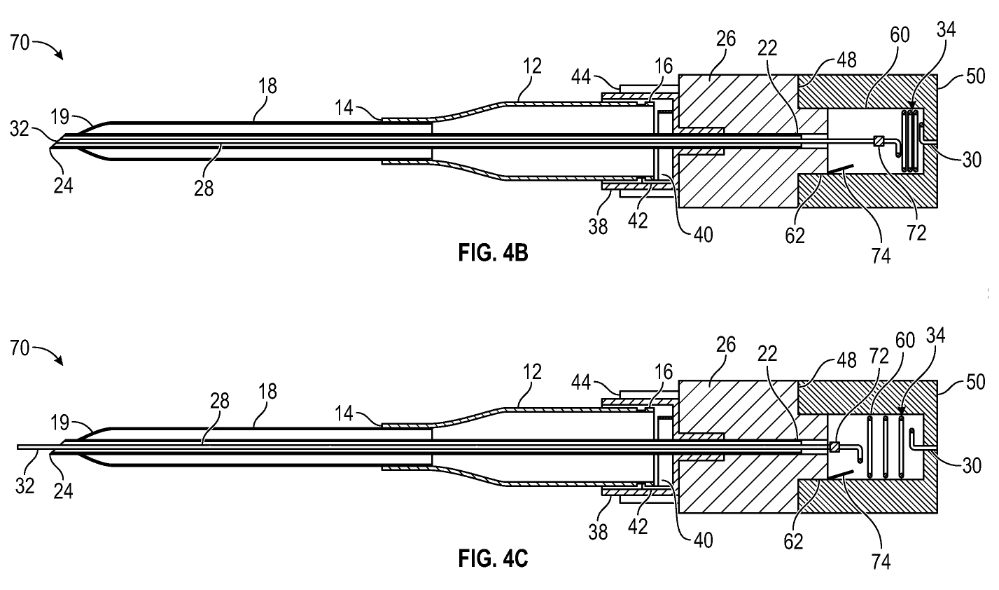

Referring now to FIGS. 4A-4C, a catheter system 70 is illustrated, according to some embodiments. In some embodiments, the catheter system 70 may be similar or identical to the catheter system 10 of FIGS. 1-3 in terms of one or more components and/or operation. In some embodiments, the guidewire 28 may include a feature 72, which may include a bump, groove, or another suitable feature. In some embodiments, the catheter system 10 may include a stop 74 contacting the feature 72 to prevent distal movement of the guidewire 28, as illustrated, for example, in FIG. 4A. In some embodiments, a distal end of the stop 74 may be coupled to a portion of the guidewire hub 36 such as, for example, the cylindrical recess 60. In some embodiments, the distal end of the stop 74 may be hingedly or pivotally coupled to the guidewire hub 36 to facilitate movement from a raised position in which the stop 74 contacts the feature 72 to a lowered position closer to a wall of the guidewire hub, illustrated, for example, in FIG. 4C.

In some embodiments, in response to compression of the coiled spring portion 34 in the proximal direction due to contact of the distal end 32 of the guidewire 28 with, for example, the skin, the tissue, the wall of the vasculature, or a particular obstacle, the stop 74 may be removed from contact with the feature 72, as illustrated, for example, in FIG. 4B. In some embodiments, in response to the stop 74 being removed from contact with the feature 72, the coiled spring portion 34 may be configured to decompress in the distal direction, as illustrated, for example, in FIG. 4C, when the sharp distal tip 24 penetrates the vasculature.

In some embodiments, the distal end 32 of the guidewire 28 may be distal to the sharp distal tip 24 a first distance, as illustrated, for example, in FIG. 4A. In some embodiments, the catheter system 10 in FIG. 4A may correspond to a resting configuration or an assembled configuration of the catheter system 10. In some embodiments, the catheter system 10 in FIGS. 4A may correspond to an insertion configuration ready for insertion into the patient. In some embodiments, in response to the stop 74 being removed from contact with the feature 72 and the coiled spring portion 34 decompressing in the distal direction, the distal end 32 of the guidewire 28 may be distal to the sharp distal tip 24 of the introducer needle 20 a second distance, as illustrated, for example, in FIG. 4C. In some embodiments, the second distance may be greater than the first distance, which may facilitate threading of the catheter 18 along the guidewire 28 after the catheter 18 is positioned within the lumen of the vasculature.

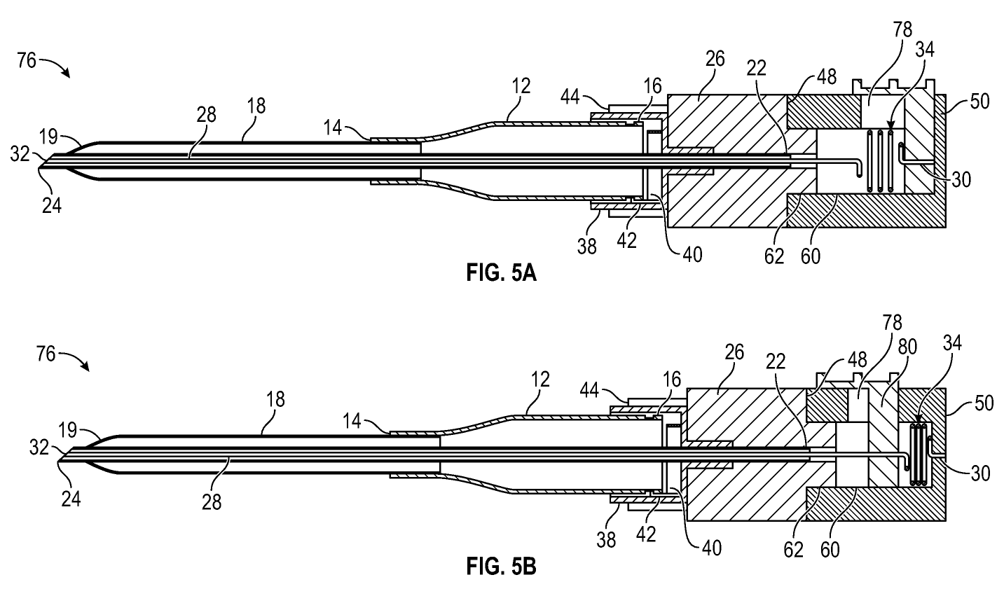

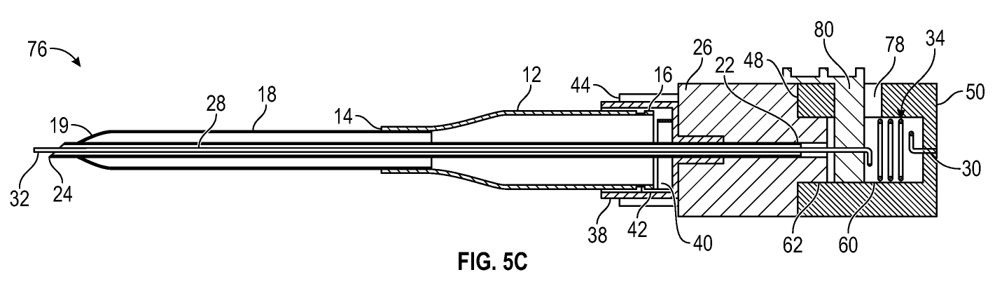

Referring now to FIGS. 5A, a catheter system 76 is illustrated, according to some embodiments. In some embodiments, the catheter system 76 may be similar or identical to the catheter system 10 of FIGS. 1-3 and/or the catheter system 70 of FIG. 4 in terms of one or more components and/or operation. In some embodiments, the guidewire hub 36 may include a slot 78. In some embodiments, the proximal end 30 of the guidewire 28 may be within the guidewire hub 36. In some embodiments, the catheter system 76 may include a guidewire slider 80 extending through the slot 78 and coupled to the guidewire 28. In some embodiments, the guidewire slider 80 may include one or more push tabs configured to contact a finger of the clinician. In some embodiments, the guidewire slider 80 may be movable along the slot 78 between a proximal position, illustrated, for example, in FIG. 5A, and a distal position.

As illustrated in FIG. 5A, in some embodiments, the guidewire slider 80 may be fixedly coupled to the guidewire 28 proximal to the coiled spring portion 34. In these and other embodiments, when the guidewire slider 80 is in the proximal position, the distal end 32 of the guidewire 28 is proximal to the sharp distal tip 24 of the introducer needle 20. In some embodiments, when the guidewire slider 80 is in the distal position, the distal end 32 of the guidewire 28 may extend distal to the sharp distal tip 24 in a resting position, but may be disposed within the introducer needle 20 due to resistance by the skin, the tissue, or the wall of the vasculature during insertion. In some embodiments, the proximal end 30 of the guidewire 28 may be secured to the guidewire slider 80.

In some embodiments, the guidewire slider 80 may provide the clinician with control over the guidewire 28, allowing the clinician to slide the guidewire slider 80 forward or distally to extend or compress the guidewire 28. In some embodiments, the clinician may also slide the guidewire slider 80 back or proximally to fully retract the guidewire 28 within the introducer needle 20 prior to insertion into the skin or during withdrawal of the guidewire 28 for a second insertion attempt. In some embodiments, the clinician may retract the distal end 32 of the guidewire 28 inside the introducer needle 20 during skin penetration by moving the guidewire slider 80 to the proximal position. In some embodiments, after the skin is penetrated, the clinician may move the guidewire slider 80 to the distal position to allow the coiled spring portion 34 to decompress when the guidewire 28 penetrates the vasculature.

In some embodiments, forward pressure applied to the guidewire slider 80 when the guidewire slider 80 is in the distal position may facilitate a tactile signal when the coiled spring portion 34 pops forward during decompression to inform the clinician of penetration of the vasculature. In some embodiments, the tactile signal when the coiled spring portion 34 pops forward may occur sooner than the blood flashback is visible to the clinician. In some embodiments, the tactile signal may be particularly helpful if the clinician’s attention is not on the catheter system 10 such as when ultrasound is being used, for example.

Referring now to FIGS. 5B-5C, in some embodiments, the guidewire slider 80 may be fixedly coupled to the guidewire 28 distal to the coiled spring portion 34. In these and other embodiments, when the guidewire slider 80 is in the proximal position, the coiled spring portion 34 may be compressed, and the distal end 32 of the guidewire 28 may be proximal to the sharp distal tip 24 of the introducer needle 20. In some embodiments, the guidewire slider 80 coupled to the guidewire 28 distal to the coiled spring portion 34 allows the clinician to retract the guidewire manually for skin penetration, after which the clinician can let go of the guidewire slider 80, automatically moving the guidewire slider 80 distally and the distal end 32 distal to the sharp distal tip 24 to allow the coiled spring portion 34 to decompress when the guidewire 28 penetrates the vasculature. In some embodiments, the proximal end 30 of the guidewire 28 may be secured within a wall of the guidewire hub 36. In some embodiments, when the guidewire slider 80 is in the distal position, the distal end 32 of the guidewire 28 may extend distal to the sharp distal tip 24 in a resting position (illustrated, for example, in FIG. 5C), but may be disposed within the introducer needle 20 due to resistance by the skin, the tissue, or the wall of the vasculature during insertion.

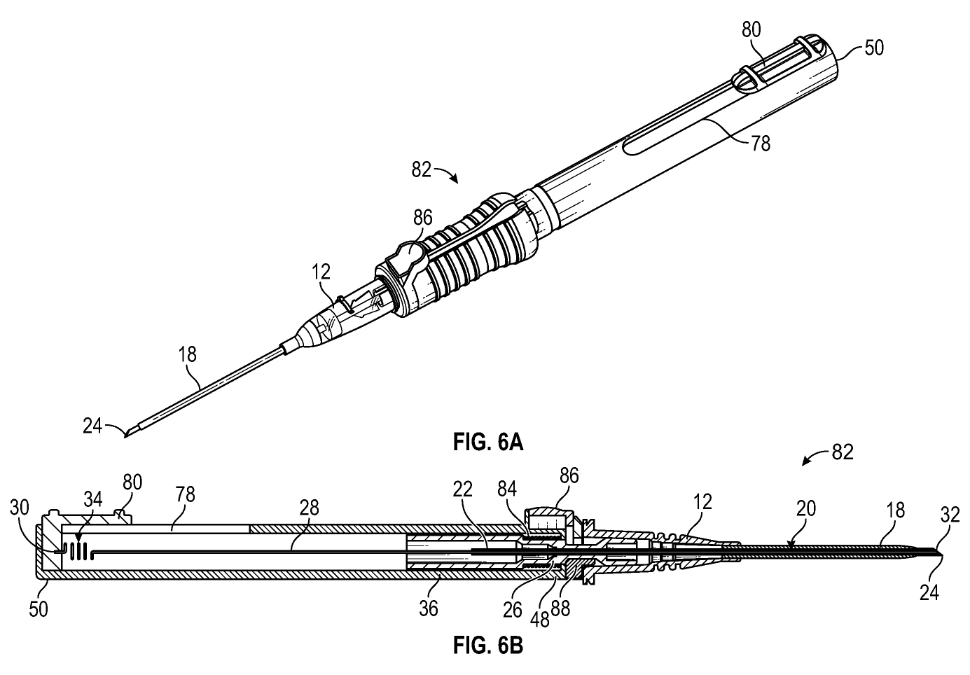

Referring now to FIGS. 6A-6B, a catheter system 82 is illustrated, according to some embodiments. In some embodiments, the catheter system 82 may be similar or identical to the catheter system 76 of FIGS. 5A in terms of one or more components and/or operation. In some embodiments, the guidewire hub 36 may include a barrel, which may be generally hollow. In some embodiments, the guidewire hub 36 may include a distal end 48 and a proximal end 50. In some embodiments, a needle hub 26 may be affixed adjacent to the proximal end 22 of the introducer needle 20 and movably disposed within the barrel. In some embodiments, a spring 84 may be disposed about the introducer needle 20 and extend between the needle hub 26 and the distal end 48 of the guidewire hub 36.

In some embodiments, an activation latch 86 having a top and a bottom may be movably mounted adjacent to the distal end 48 of the guidewire hub 36. In some embodiments, the activation latch 86 may be adapted for selective engagement with the needle hub 26 to hold the needle hub 26 adjacent to the distal end 48 of the guidewire hub 36 against the bias of the spring 84 such that the introducer needle 20 extends beyond the distal end 48 of the guidewire hub 36 and through the catheter 18 with the catheter hub or adapter 12 adjacent to the distal end 48 of the guidewire hub 36. In some embodiments, a projection 88 may extend from the activation latch 86 for engagement with the catheter adapter 12 to prevent movement of the activation latch 86 when the catheter adapter 12 is adjacent to the distal end 48 of the guidewire hub 36. The activation latch 86 and the projection 88 may be further described in U.S. Patent No. 5,501,675, filed December 27, 1994, and entitled “SAFETY CATHETER HAVING SAFETY STOP PUSH BUTTON,” which is hereby incorporated by reference in its entirety.

In some embodiments, the guidewire hub 36 may include a slot 78. In some embodiments, the proximal end 30 of the guidewire 28 may be within the guidewire hub 36. In some embodiments, the catheter system 82 may include a guidewire slider 80 extending through the slot 78 and coupled to the guidewire 28. In some embodiments, the guidewire slider 80 may include one or more push tabs configured to contact a finger of the clinician. In some embodiments, the guidewire slider 80 may be movable along the slot 78 between a proximal position, illustrated, for example, in FIGS. 6A-6B, and a distal position.

As illustrated in FIGS. 6A-6B, in some embodiments, the guidewire slider 80 may be fixedly coupled to the guidewire 28 proximal to the coiled spring portion 34. In these and other embodiments, when the guidewire slider 80 is in the proximal position, the distal end 32 of the guidewire 28 may be proximal to the sharp distal tip 24 of the introducer needle 20. In some embodiments, when the guidewire slider 80 is in the distal position, the distal end 32 of the guidewire 28 may extend distal to the sharp distal tip 24 in a resting position but may be disposed in the introducer needle 20 due to resistance by the skin, the tissue, or the wall of the vasculature during insertion. In some embodiments, the proximal end 30 of the guidewire 28 may be secured to the guidewire slider 80.

In some embodiments, the guidewire slider 80 may provide the clinician with control over the guidewire 28, allowing the clinician to slide the guidewire slider 80 forward or distally to extend or compress the guidewire 28. In some embodiments, the clinician may also slide the guidewire slider 80 back or proximally to fully retract the guidewire 28 within the introducer needle 20 prior to insertion into the skin or during withdrawal of the guidewire 28 for a second insertion attempt. In some embodiments, the clinician may retract the distal end 32 of the guidewire 28 inside the introducer needle 20 during skin penetration by moving the guidewire slider 80 to the proximal position. In some embodiments, after the skin is penetrated, the clinician may move the guidewire slider 80 to the distal position to allow the coiled spring portion 34 to decompress when the guidewire 28 penetrates the vasculature. In some embodiments, after the coiled spring portion 34 decompresses when the guidewire 28 penetrates the vasculature, the catheter adapter 12 may be advanced distally and then the activation latch 86 may be depressed.

In some embodiments, forward pressure applied to the guidewire slider 80 when the guidewire slider 80 is in the distal position may facilitate a tactile signal when the coiled spring portion 34 pops forward during decompression to inform the clinician of penetration of the vasculature. In some embodiments, the tactile signal when the coiled spring portion 34 pops forward may occur sooner than the blood flashback is visible to the clinician. In some embodiments, the tactile signal may be particularly helpful if the clinician’s attention is not on the catheter system 10 such as when ultrasound is being used, for example.

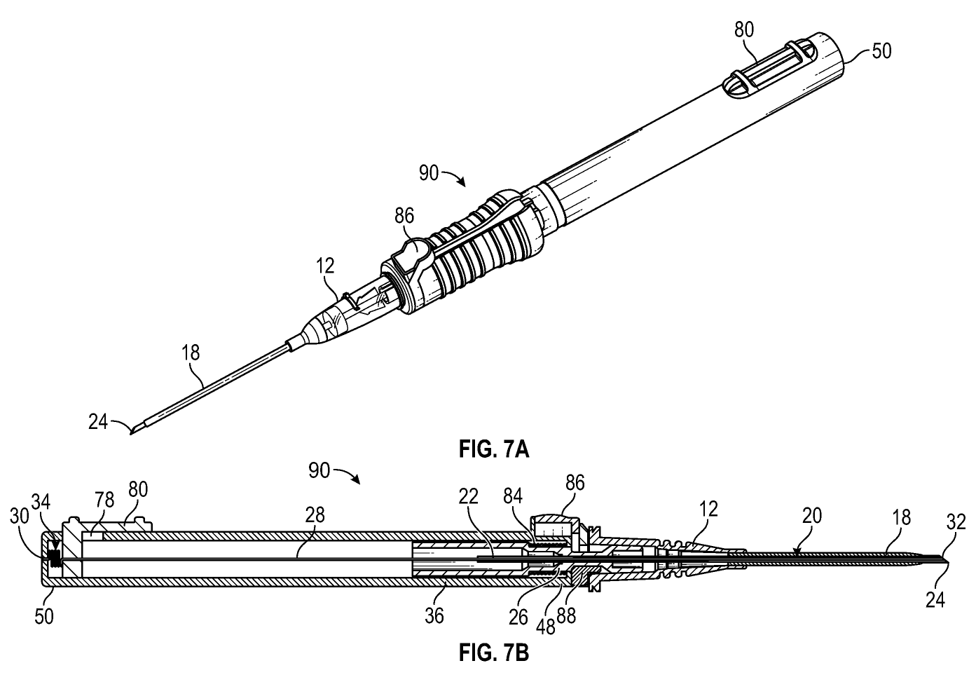

Referring now to FIGS. 7A-7B, a catheter system 90 is illustrated, according to some embodiments. In some embodiments, the catheter system 90 may be similar or identical to the catheter system 76 of FIGS. 5B-5C and/or the catheter system 82 of FIGS. 6A-6B in terms of one or more components and/or operation. In some embodiments, the guidewire slider 80 may be fixedly coupled to the guidewire 28 distal to the coiled spring portion 34. In these and other embodiments, when the guidewire slider 80 is in the proximal position, the coiled spring portion 34 may be compressed, and the distal end 32 of the guidewire 28 may be proximal to the sharp distal tip 24 of the introducer needle 20.

In some embodiments, the guidewire slider 80 coupled to the guidewire 28 distal to the coiled spring portion 34 allows the clinician to retract the guidewire manually for skin penetration, after which the clinician can let go of the guidewire slider 80, automatically moving the guidewire slider 80 distally and the distal end 32 distal to the sharp distal tip 24 to allow the coiled spring portion 34 to decompress when the guidewire 28 penetrates the vasculature. In some embodiments, the proximal end 30 of the guidewire 28 may be secured within a wall of the guidewire hub 36. In some embodiments, when the guidewire slider 80 is in the distal position, the distal end 32 of the guidewire 28 may extend distal to the sharp distal tip 24 in a resting position (similar to FIG. 5C), but may be disposed within the introducer needle 20 due to resistance by the skin, the tissue, or the wall of the vasculature during insertion.

All examples and conditional language recited herein are intended for pedagogical objects to aid the reader in understanding the invention and the concepts contributed by the inventor to furthering the art and are to be construed as being without limitation to such specifically recited examples and conditions. Although embodiments of the present inventions have been described in detail, it should be understood that the various changes, substitutions, and alterations could be made hereto without departing from the spirit and scope of the invention.

1. A catheter system, comprising:
a catheter adapter comprising a distal end and a proximal end;
a catheter comprising a distal end, wherein the catheter extends distally from the distal end of the catheter adapter;
an introducer needle comprising a proximal end and a sharp distal tip, wherein the introducer needle extends through the catheter;
a needle hub, wherein the proximal end of the introducer needle is fixed within the needle hub;
a guidewire comprising a proximal end, a distal end, and a coiled spring portion between the proximal end of the guidewire and the distal end of the guidewire, wherein the guidewire is disposed within the introducer needle, wherein the coiled spring portion is proximal to the sharp distal tip; and
a guidewire hub proximal to the needle hub, wherein the proximal end of the guidewire is fixed within the guidewire hub.

2. The catheter system of claim 1, wherein the coiled spring portion is configured to compress in response to the distal end of the guidewire contacting a blood vessel and decompress in response to entry of the guidewire into the blood vessel.

3. The catheter system of claim 2, wherein the distal end of the guidewire is distal to the sharp distal tip, wherein the distal end of the guidewire is configured to retract proximal to the sharp distal tip in response to compression of the coiled spring portion.

4. The catheter system of claim 3, wherein the guidewire hub is coupled to the needle hub, wherein in response to uncoupling of the guidewire hub and the needle hub and proximal movement of the guidewire hub with respect to the needle hub, the distal end of the guidewire is configured to retract proximal to the sharp distal tip.

5. The catheter system of claim 4, wherein a proximal end of the needle hub comprises first cutout and a distal end of the guidewire hub comprises a second cutout, wherein the first cutout and the second cutout are proximate each other to form a pocket having a U-shape configured to receive a thumb or finger of a user.

6. The catheter system of claim 4, wherein the guidewire hub is coupled to the needle hub in a slip fit.

7. The catheter system of claim 6, wherein a distal end of the guidewire hub comprises a cylindrical recess, wherein a proximal end of the needle hub comprises a cylindrical protrusion that is disposed within the cylindrical recess.

8. The catheter system of claim 1, wherein the coiled spring portion is proximal to the proximal end of the introducer needle.

9. The catheter system of claim 1, wherein the coiled spring portion is disposed within the guidewire hub.

10. The catheter system of claim 1, further comprising a needle safety housing coupled to the proximal end of the catheter adapter, further comprising a needle safety clip disposed within the needle safety housing and configured to shield the sharp distal tip when the sharp distal tip is withdrawn proximally past the needle safety clip.

11. The catheter system of claim 1, wherein the guidewire comprises a marking, further comprising a transparent window, wherein in response to the coiled spring portion being compressed, the marking is configured to move into or out of the transparent window.

12. The catheter system of claim 1, wherein the distal end of the guidewire is straight.

13. The catheter system of claim 1, wherein the distal end of the guidewire comprises a coil distal to the sharp distal tip.

14. The catheter system of claim 1, wherein the guidewire comprises a feature, further comprising a stop contacting the feature to prevent distal movement of the guidewire, wherein in response to compression of the coiled spring portion, the stop is removed from contact with the feature and the coiled spring portion is configured to decompress.

15. The catheter system of claim 14, wherein the distal end of the guidewire is distal to the sharp distal tip a first distance, wherein in response to the stop being removed from contact with the feature and the coiled spring portion decompressing, the distal end of the guidewire is distal to the sharp distal tip a second distance, wherein the second distance is greater than the first distance.

16. A catheter system, comprising:
a catheter adapter comprising a distal end and a proximal end;
a catheter comprising a distal end, wherein the catheter extends distally from the distal end of the catheter adapter;
an introducer needle comprising a proximal end and a sharp distal tip, wherein the introducer needle extends through the catheter;
a needle hub, wherein the proximal end of the introducer needle is fixed within the needle hub;
a guidewire comprising a proximal end, a distal end, and a coiled spring portion between the proximal end of the guidewire and the distal end of the guidewire, wherein the guidewire is disposed within the introducer needle, wherein the coiled spring portion is proximal to the distal end of the catheter;
a guidewire hub proximal to the needle hub, wherein the guidewire hub comprises a slot, wherein the proximal end of the guidewire is within the guidewire hub; and
a guidewire slider extending through the slot and coupled to the guidewire, wherein the guidewire slider is movable along the slot between a proximal position and a distal position.

17. The catheter system of claim 16, wherein the guidewire slider is coupled to the guidewire proximal to the coiled spring portion, wherein the proximal end of the guidewire is secured to the guidewire slider.

18. The catheter system of claim 17, wherein when the guidewire slider is in the proximal position, the distal end of the guidewire is proximal to the sharp distal tip.

19. The catheter system of claim 16, wherein the guidewire slider is coupled to the guidewire distal to the coiled spring portion, wherein the proximal end of the guidewire is secured within a wall of the guidewire hub.

20. The catheter system of claim 19, wherein when the guidewire slider is in the proximal position, the spring is compressed and the distal end of the guidewire is proximal to the sharp distal tip.
